(19) United States
(12) Patent Application Publication
(10) Pub. No.: US 2002/0082537 A1
(43) Pub. Date: Jun. 27, 2002

(54) Hip and shoulder wrap

(76) Inventor: Doris MacAllister, N. Quincy, MA (US)

Correspondence Address: D Michael Burns, 37-6 Belmont Court, Brockton, MA, 02301, US

(21) Appl. No.: 09/735,965

(22) Filed: Dec. 13, 2000

Related U.S. Application Data
(60) Provisional application No. 60/173,107, filed on Dec. 27, 1999.

(51) Int. Cl. A61F 5/00

(52) U.S. Cl. 602/4; 602/20

(57) ABSTRACT

A therapeutic wrap and method for stabilizing a hip or a shoulder. The wrap comprises an elongated, flexible, relatively thin (⅛ inch) fabric strap that is secured around the hip and stabilized by wrapping and securing around the adjacent upper thigh region of the patient. The wrap having a movable pocket containing a magnet pad for strategically positioning so as to be juxtaposed with the injured hip or shoulder. The wrap is fastened in place by hook and loop closure system as is the movable pocket. The wrap is universal and can be used on either leg or shoulder by merely adjusting the length. The length of the wrap directly proportional to the body size of the patient.

### CROSS REFERENCE TO RELATED APPLICATION

This application is based on Provisional Patent Application Ser. No. 60/173,107, filed Dec. 27, 1999.

### BACKGROUND OF THE INVENTION

1. Field of the Invention

The present invention relates to the field of therapeutic devices and wraps. More particularly, the present invention relates to the field of therapeutic wraps used to either stabilize the hip in its proper position to prevent hip rotation, or to support the shoulder while alleviating pain from subluxation, rotator-cuff or joint compression.

2. Description of the Prior Art

It is well known that for years people have suffered needlessly from hip pain. The only relief an ailing person could get for hip pain was either from pills or surgery. As people grow older, the structure and alignment of the hips can change. This change may be caused by hip rotation, which occurs when the femur bone and the hip socket are malaligned. This condition may be the result from injury, loss of bone density, arthritis or just the process of getting older. As the condition worsens, the results can be pain, loss of mobility and general discomfort. The condition may be the result of the hip rotating and becoming out of alignment with the rest of the body, thereby creating other aches and pains in the back and knees from overcompensating from hip pain. The only relief was hip replacement surgery.

The proper alignment of the hips is very important to a person. If the condition worsens, the hip rotates, pain develops, the lower back is affected and possibly the knees as well.

The prior art has taught of hip stabilizers such as casts or other bulky types comprising corsets or belts usually with metal biasing structures for support between a hip wrap and the thigh wrap. It was often that the bulkiness of the device obviated the problem of atrophy of the muscles and the deterioration of joints caused be complete immobilization of muscles and joints while a hip was stabilized.

U.S. Pat. No. 4,905,678 issued to Cumins et al, on Mar. 6, 1990, teaches of a hip stabilizer that includes a semi-rigid belt and at least one respective hip pad with means for biasing an abduction.

U.S. Pat. No. 5,620,412 issued to Modglin on Apr. 15, 1997, shows a substantial rigid bi-valve type abduction system for supporting the hip joint. The system includes a pelvic girdle and leg flanges to prevent migration thereof on the patient.

The latest devices are reflective of patents such as U.S. Pat. No. 5,419,755 issued to Fukumoto et al. on May 30, 1995. This as a weight bearing brace for a person having hip joint trouble. It incorporates: an upper socket removably attached to the periphery of the upper thigh; a lower socket removably attached to the periphery of the lower thigh; and connected there between.

Another hip stabilization device is illustrated in U.S. Pat. No. 4,481,941 issued to Rolfes on Nov. 13, 1984, wherein Rolfes teaches the use of a hinged brace held in alignment with the hip joint by a belt adjustably engaged about the patient's waist. The brace prevents rotation of the hip joint and lateral motion of the leg.

Most of the prior art patents that teach the use of fabric type wraps are basically directed towards post-operative use. Such surgical hip wraps are disclosed by Pat. Nos. 4,977,893, 5,425,702 and 5,840,050. These would appear to be closest to the present invention, and they all use hook and loop material to fasten relatively soft fabric about the waist and thigh of the patient. None teach of the manner in which the device is wrapped to prevent rotation of the hip as being of the utmost importance.

While most of the discussion of the prior art relates to devices for supporting the hip, it is to be appreciated that the present invention primarily seeks to solve a much needed solution for relieving hip pain, however the device of the present invention can also be used to ease shoulder discomfort and pain as well.

None of the above inventions and Patents, taken either singly or in combination, is given to describe the instant invention as claimed.

### SUMMARY OF THE INVENTION

The present invention provides for a hip or shoulder wrap herein also referred to as WRAP'PRENE™ to enable a patient to stabilize and support a hip or shoulder while contributing to proper alignment, thereby relieving pain. The device of the present invention is a non-invasive method to effectively reduce pain and it is both economical as well as easy to use.

WRAP'PRENE™ is made from a neoprene strip with nylon on one side. The nylon allows the neoprene to stretch. The length varies depending on the size of the patient. Normal ranges of size when using as a hip wrap would be 76 to 110 inches, with the width being 6 inches and the thickness about ⅛ of an inch. The thickness of the wrap is important so that flexibility is provided without loss of health benefits. When the wrap is used on the shoulder area, the length is between 50 to 75 inches, with the width being 4 inches. The lack of bulkiness allows the wrap to be worn comfortably under clothes. The ends of the wrap are rounded to reduce the tendency of the corners peeling back. The means of fastening the wrap in place is by hook and loop. The opposing side to the nylon is loop material and both ends have the hook material. Each end of the wrap has a 6 inch piece of hook material with a pair of hook strips giving added fastening support. The effect of the elasticity of the neoprene combined with the proper wrapping process supporting the joint gives relief to patients. In addition the neoprene promotes warmth and blood flow that aids in the healing process.

WRAP'PRENE™ is designed with a movable pocket strategically positioned by hook and loop fastening system on the hip or the shoulder to house optional magnets. The magnets will decrease the inflammation. The magnet combines the effect of inflammation-fighting therapy with the support and benefit of neoprene. The primary target for WRAP'PRENE™ is to promote proper alignment in the hips that are either externally rotated or internally rotated. It can be very easily donned or doffed by the patient. When used to give shoulder relief, WRAP'PRENE™ alleviates pain from subluxation, rotator-cuff injury or joint compression. The pocket for the magnet is positioned so that it will be on the acromion process of the scapular.

An object of the present invention is to offer relief to people suffering from hip pain caused by arthritis or trauma causing incorrect alignment of a hip.

Another object of the present invention is to give shoulder support to help alleviate pain from subluxation, rotator-cuff injury or joint compression.

An object of the present invention is to promote warmth and blood flow from the use of neoprene, thereby aiding in the healing process.

Another object is to decrease the inflammation by the use of magnets thereby combining the effect of inflammation-fighting therapy with the support and benefits of the neoprene.

An object of the present invention is to provide an affordable alternative to surgery.

Yet still an object of the present invention is to provide a device having no bulky or rigid parts such that it can very easily be donned or doffed by the patient, and also worn comfortably under clothes.

These and other objects of the present invention will become readily apparent upon further review of the following specification and drawings.

### BRIEF DESCRIPTION OF THE DRAWINGS

FIG. 1 is a frontal planar view of the device of the present invention.

FIG. 2 is a top view of the embodiment of FIG. 1.

FIG. 3 is a pictorial view of the embodiment of FIG. 1 illustrating its operation and method of attachment to a patient's right hip for hip support and prevention of external hip rotation.

FIG. 4 is a pictorial view of the embodiment of FIG. 1 illustrating its operation and method of attachment to a patient's right hip for hip support and prevention of internal hip rotation.

FIG. 5 is a pictorial view of the embodiment of FIG. 1 illustrating its operation and method of attachment to a patient's left shoulder for support and relief of pain.

### DETAILED DESCRIPTION OF THE PREFERRED EMBODIMENT

As people grow older, the structure and alignment of their hips can change. This change may be caused by hip rotation. Hip rotation occurs when the femur bone and the hip socket are malaligned. This condition may be the result from injury, loss of bone density, arthritis or just the process of aging. As the condition worsens, pain, loss of mobility and general discomfort may occur. This condition may be the result of the hip rotating and becoming out of alignment with the rest of the body creating other aches and pains in the back and knees from overcompensating due to the hip pain. As of now, the only relief was hip replacement surgery.

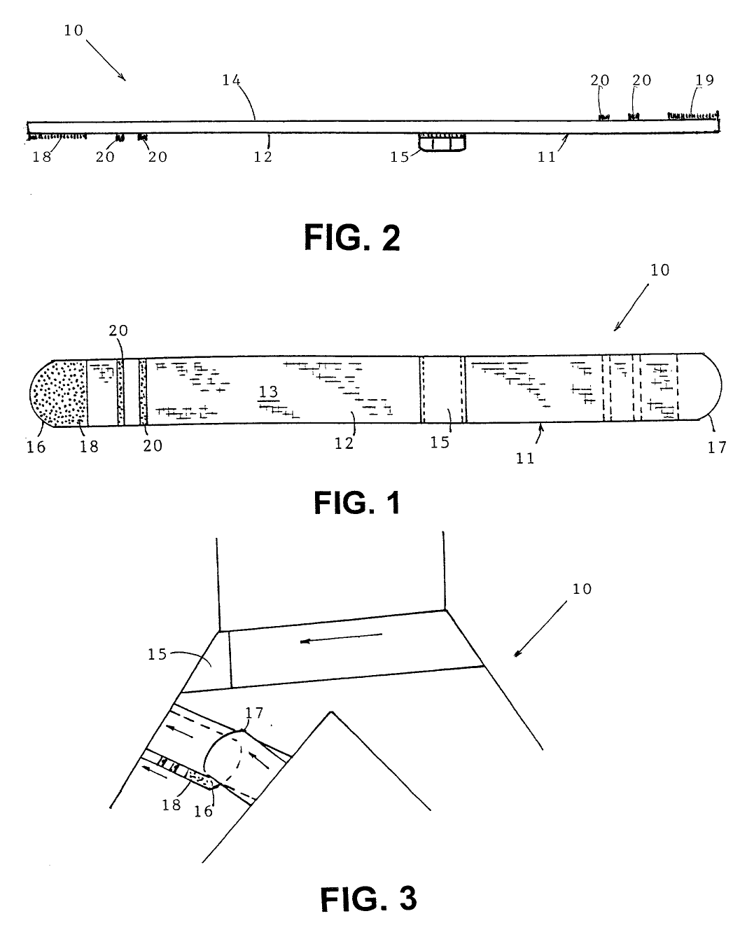

Referring to the drawings for a clearer understanding of the invention, it should be noted in FIGS. 1 and 2 that the present invention contemplates the use of a flexible wrap 10, also referred to as WRAP'PRENE™, to relieve the pain caused by a hip not being in proper alignment, or in the case of shoulder relief for supporting the shoulder while alleviating pain from subluxation, rotator-cuff injury or joint compression. The wrap 10 promotes proper alignment to a hip and prevents either external or internal rotation therein, which will depend on the needs of the patient. These non-invasive methods effectively reduce pain and can be worn comfortably under clothes.

The wrap 10 as shown in FIGS. 1 and 2 is comprised of a single flexible, flat, washable neoprene strap 11 having a front side 12 which is primarily covered with loop material 13 (i.e. VELCRO, a trademark for a fabric employing J-hooks and loops to fasten) and a rear side 14 of nylon material. Neoprene material holds heat and the warmth maintains blood flow to bring oxygen to healing tissues. The thickness of strap 11 is a consideration and it should be about ⅛ of an inch to allow wrap 10 a degree of flexibility without loss of health benefits and also to enable the patient to wear wrap 10 under street clothes. The nylon material allows the neoprene to stretch. The effect of the elasticity of the neoprene combined with the normal wrapping process, in which the neoprene promotes warmth and blood flow, aids in the healing process. The dimensions of wrap 10 will vary with use and patient size. For hip stabilization wrap 10 will be 6 inches wide and between 76 to 110 inches in length. For use as a shoulder support wrap 10 will be 4 inches wide and the length between 50 to 75 inches. Each wrap 10 will have a movable pocket 15 which can be strategically positioned on strap 11 by hook and loop fastening means for the purpose of holding a magnetic pad (not shown but to be contained within pocket 15). The use of magnets may be optional but their therapeutic value is well known. The effects of the magnet pad are two-fold. It will promote healing by stimulating blood flow to the affected region, which brings extra nutrients and reduces inflammation as toxins are flushed out. Magnetic therapy also decreases pain by reducing the sensitivity of the nerve endings. The magnet combines the effects of inflammation fighting therapy and pain relief with the support and benefit of neoprene. The location of pocket 15 should be such that when wrap 10 is used on the patient's hip, pocket 15 will be thereby positioned between the anterior iliac spine and anterior inferior iliac spine of the patient. Pocket 15 will be about 6 inches wide and 3.5 inches in length. When wrap 10 is used for shoulder relief, pocket 15 should be positioned such that it will be at the acromion process of the scapula of the patient. Pocket 15 will be 3 inches wide by 3.5 inches in length.

Strap 11 is comprised of opposing rounded ends, a first end 16 and a second end 17. Ends 16 and 17 being rounded to avoid comers that have a tendency to peel back. First end 16 having a hook section 18 located on the front side 12 of strap 11 while second end 17 having a hook section 19 on rear side 14. Each end 16 and 17 having a pair of reinforcing hook strips 20 in close proximity to their respective hook sections 18 and 19. Strips 20 being approximately one inch in width.

A hip may rotate either outward or inward. Wrap 10 can be used for either situation, whether it be for preventing external or internal hip rotation, but it is only as good as the manner in which it is used. The inventive concept of the present invention is directly proportional to the wrapping method as illustrated in FIGS. 3-5.

FIG. 3 depicts wrap 10 preventing external rotation of the right hip of a patient. The method steps are as follows: first, placing the first end 16 on the inside of the thigh with front side 12 facing away from the patient; then, winding wrap 10 counter-clockwise (as viewed in the drawing) twice around the thigh; then bringing wrap 10 diagonally up to, over and around the injured hip; then, taking wrap 10 to and around the uninjured hip; then, diagonally crossing the patient's abdomen; and finally, bringing second end 17 around to the medial thigh, whereby rear side hook section 19 is fastened to loop material 13 of front side 12. Hook strips 20 strengthen the attachment, while movable pocket 15 holding the magnet is adapted to be in its proper position, which is between the anterior iliac spine and anterior inferior iliac spine area of the patient.

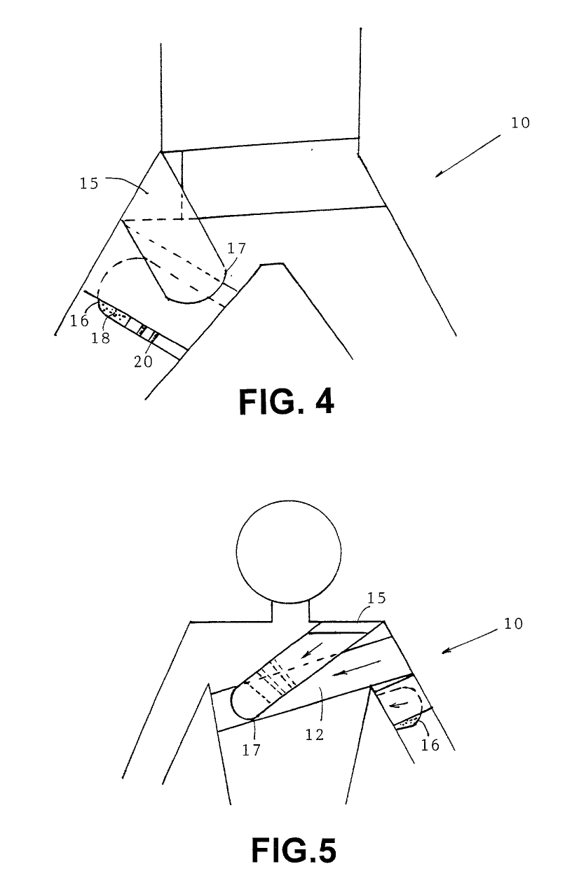

FIG. 4 depicts wrap 10 being used to prevent internal rotation of the injured right hip of a patient. The method starts with first end 16 positioned on the outside of the thigh, again with front side 12 facing away from the patient; then winding wrap 10 is clockwise twice around the thigh; then diagonally crossing wrap 10 about the abdomen where it is brought up and over the uninjured hip; then, crossing over and around the injured hip; and finally, attaching the wrap together by fastening rear side hook section 19 to loop material 13 on the anterolateral side of the thigh.

FIG. 5 depicts wrap 10 being utilized to support a patient's left shoulder. The method starts with first end 16 being placed on the inside of the left arm with loop material 13 facing away from the patient; then winding wrap 10 around the arm twice in a counter-clockwise direction; then taking wrap 10 across the chest and under the underarm of the uninjured right side; then taking it diagonally up to and across the back; and finally up and over the injured right shoulder before hook material 19 of second end 17 attaches to loop material 13 in area of close proximity to the patient's chest. Pocket 15 holding the magnet pad is adapted to be in on the scapula area of the patient.

It is to be understood that the present invention is not limited to the sole embodiments described above, but encompass any and all embodiments within the scope of the following claims.

I claim:

1. A therapeutic hip and shoulder wrap for alleviating pain in a patient, said wrap comprising:
a uniform strap of flexible, flat, neoprene material of indeterminable length, the strap having two sides, a front side and a rear side, the rear side having a backing of nylon material to provide a measure of elasticity to the strap;
the strap having opposing ends, a first end and a second end, the ends being rounded to avoid having corners that may have a tendency to peel back;
fastening means comprising of hook and loop material; and
a movable pocket holding a magnet pad adapted so as to be strategically positioned on the strap by hook and loop fastening means, the magnet pad used to promote healing by stimulating blood flow to the hip or shoulder, which brings extra nutrients and reduces inflammation as toxins are flushed out, the magnet pad also decreasing pain by reducing sensitivity of the nerve endings.

2. The therapeutic wrap according to claim 1, wherein the lengths of the wrap when used for hip relief are generally between 76 to 110 inches, depending on the body size of the patient, the width of the wrap being about 6 inches, and the magnet pocket approximately 6 inches in width and 3.5 inches in length.

3. The therapeutic wrap according to claim 2, wherein the magnet pocket is adapted so as to be strategically positioned on the strap such that it will be located between the anterior iliac spine and the anterior inferior iliac spine of the hip bone, when the wrap is in position on the patient.

4. The therapeutic wrap according to claim 1, wherein the lengths of the wrap when used for alleviating shoulder pain are generally between 50 and 75 inches, depending on the body size of the patient, the width of the wrap being about 4 inches, and the magnet pocket approximately 3½ inches in width and 3 inches in length.

5. The therapeutic wrap according to claim 4, wherein the movable magnet pocket is adapted so as to be strategically positioned on the strap such that it will be located on the acromion process of the scapula, when the wrap is in position.

6. The therapeutic wrap according to claim 1, wherein the hook and loop fastening means comprises:
loop fastening material on the front side of the strap; and
hook fastening material on the front side at the first end of the strap, and on the rear side of the strap at the second end, the hook fastening material comprising of a pad approximately 6 inches in length and a pair of 1 inch reinforcing strips.

7. A therapeutic hip and shoulder wrap for alleviating pain in a patient, the wrap comprising a uniform strap of flexible, flat, neoprene material of indeterminable length, the strap having two sides, a front side and a rear side;
the strap having opposing ends, a first end and a second end, the ends being rounded to avoid having corners that may have a tendency to peel back;
fastening means, the means comprising of hook and loop materials, loop material generally disposed on the front side of the strap therein, hook material generally on the front side of the first end and on the rear side of the second end; and
a movable magnet pocket positioned on the strap by hook and loop fastening means, the pocket housing a magnet pad for promoting healing by stimulating blood flow to the hip or shoulder;
whereby means for alleviating pain or discomfort are available, the means to ease pain caused by external rotation of a patient's hip may be relieved by a method comprising the steps:
placing the first end of the wrap on the inside of a patient's thigh with the front side facing outwardly;
winding the wrap in a counter-clockwise direction twice around the thigh;
bringing the wrap diagonally up to, then over and around the patient's injured hip, such that the magnet pad is thereby adapted so as to be in a juxtaposed position between the anterior iliac spine and the anterior inferior iliac spine;
winding the wrap across and around the patient's uninjured hip and then diagonally back across the patient's abdomen; and
finally, fastening the wrap at the medial thigh by attaching the hook material at the second end to the loop material on the front side of the strap.

8. The therapeutic wrap according to claim 7, wherein the means for alleviating pain and discomfort which are caused by internal rotation of a patient's hip are accomplished by a method comprising the steps:
placing the first end of the wrap on the outside of the patient's thigh with the front side facing outwardly;
winding the wrap in a clockwise direction twice around the thigh;
bringing the wrap diagonally up and across the patient's abdomen where it is then brought up and over the patient's uninjured hip;
extending the wrap across, over and around the patient's injured hip, whereby the magnet pad is adapted to be in a juxtaposed position between the anterior iliac spine and the anterior inferior iliac spine; and
finally, fastening the wrap on the anterolateral side of the thigh by attaching the rear side hook section of the strap to the loop material on the front side of the strap.

9. The therapeutic wrap according to claim 7, wherein the means for alleviating pain and discomfort which are caused by shoulder pain are accomplished by a method comprising the steps:
placing the first end of the wrap on the inside of a patient's arm, the arm extending from an injured shoulder, the front side of the strap facing outwardly;
winding the wrap is in a counter-clockwise direction twice around the arm;
bringing the wrap across the chest and under the underarm of the patient's uninjured shoulder;
taking the wrap diagonally up and across the patient's back, then over the injured shoulder, whereby the magnet is adapted to be in a juxtaposed position on the acromion process of the scapula; and
finally, fastening the wrap on the chest area of the patient by attaching the hook material on the second end to the loop material on the front side of the strap.

10. The therapeutic wrap according to claim 7, wherein the lengths of the wrap when used for hip relief are generally between 76 and 110 inches depending on the patient body size, the width of the wrap being about 6 inches, and the magnet pocket approximately 6 inches in width and 3.5 inches in length.

11. The therapeutic wrap according to claim 7, wherein the lengths of the wrap when used to alleviate shoulder pain are generally between 50 and 75 inches, depending on the patient body size, the width of the wrap being about 4 inches, and the magnet pocket approximately 3.5 inches in width and 3.0 inches in length.

12. A method for applying a therapeutic wrap to a patient for alleviating pain due to hip malalignment caused by external hip rotation, the patient having two opposing hips, one hip being injured, and a leg extending from the injured hip, the method comprising the steps of:
placing a first end of the wrap on the inside of the leg with a loop material on one side of the wrap facing outwardly;
winding the wrap in a counter-clockwise direction twice around the leg;
bringing the wrap diagonally up to, and over and around the injured hip, whereby a movable magnet pad is adapted by hook and loop fastening means to be strategically positioned on the wrap, so as to be in a juxtaposed relationship between the anterior iliac spine and the anterior inferior iliac spine;
winding the wrap across and around the uninjured hip and then diagonally back across the abdomen; and
finally, fastening the wrap at the medial side of the leg by means of hook and loop material.

13. A method for applying a therapeutic wrap to a patient for alleviating pain due to hip malalignment caused by internal hip rotation, the patient having two opposing hips, one hip being injured, and a leg extending from the injured hip, the method comprising the steps:
placing a first end of the wrap on the outside of the leg with a loop material on one side of the wrap facing outwardly;
winding the wrap in a clockwise direction twice around the leg;
bringing the wrap diagonally up and across the abdomen where it is then brought up and over the uninjured hip;
extending the wrap across to, over and around the injured hip, whereby a movable magnet pad is strategically positioned on the wrap by hook and loop fastening means so as to be in a strategically disposed within the wrap is positioned in a juxtaposed relationship between the patient's anterior iliac spine and the patient's anterior inferior iliac spine; and
finally, fastening the wrap on the patient's anterolateral side of the leg by means of hook and loop material.
